(19) United States
(12) Patent Application Publication
(10) Pub. No.: US 2023/0268069 A1
(43) Pub. Date: Aug. 24, 2023

(54) METHOD FOR MONITORING A MEDICAL INTERVENTION, MONITORING SYSTEM AND COMPLETE SYSTEM

(71) Applicant: Siemens Healthcare GmbH, Erlangen (DE)

(72) Inventors: Marcus Pfister, Bubenreuth (DE); Philipp Roser, Erlangen (DE); Christian Kaethner, Freiburg (DE)

(21) Appl. No.: 18/113,620

(22) Filed: Feb. 23, 2023

(30) Foreign Application Priority Data
Feb. 24, 2022 (DE) 10 2022 201 914.8

(51) Int. Cl. G16H 40/63

(52) U.S. Cl. CPC G16H 40/63

(57) ABSTRACT

A method for monitoring a medical intervention taking place in an operating room or examination room, or a medical examination on a patient by medical personnel includes acquiring sensor data from at least one acoustic sensor, of at least one individual among the medical personnel during the medical intervention or the medical examination. The sensor data is evaluated as regards recognition of stress and/or negative emotions of the at least one individual. When stress and/or negative emotions are recognized, at least one cause of the stress and/or of the negative emotions is determined using the sensor data and/or further optical, acoustic, haptic, digital, and/or other data acquired during the medical intervention or the medical examination. Measures are triggered to remediate the determined cause and/or to reduce the stress and/or the negative emotions of the at least one individual.

This application claims the benefit of German Patent Application No. DE 10 2022 201 914.8, filed on Feb. 24, 2022, which is hereby incorporated by reference in its entirety.

### BACKGROUND

The present embodiments relate to monitoring a medical intervention taking place in an operating room or examination room, or a medical examination on a patient by medical personnel.

Current research topics in medical engineering are concerned with, among other things, the topic of “Digitization in the OP,” and with equipping an operating room with sensors (e.g., with three-dimensional (3D) cameras or microphones). Data from these sensors may be used, for example, to draw conclusions about a current step in an operational intervention (N. Padoy et al.: “Machine and deep learning for workflow recognition during surgery,” Minimally Invasive Therapy & Allied Technologies, 2019) or to facilitate operation of a device (e.g., a medical imaging device or medical robot system). One example of an improvement in the operation of a device is the control of the device by voice input. A corresponding known field of research is “Natural Language Processing (NLP),” (e.g., the recognition of speech in the spoken context).

Even with these new topics, the performance of a minimally invasive procedure or the operation of a device such as, for example, a C-arm X-ray device or a medical robot system remains a complex task. Many aspects contribute to increased stress on the part of one or more individuals among the medical personnel who are performing the intervention and/or operating the device. This leads to an increased susceptibility to errors in the procedure.

This also applies, for example, for remotely executed interventions that are performed (e.g., via a robot system) by an expert who is located outside the hospital in question, frequently even in a different city or a different country, and is connected by a data stream. Both the geographical separation and the often fairly low level of experience of the medical personnel present on site may result in corresponding stressful situations. Further, the “experience level” of such a team or also the correspondingly complex operational procedures themselves may also correspondingly contribute to this.

It is known in connection with voice recognition for emotions and stress also to be recognized from voice recordings (see, e.g., “Emotion recognition from speech: a review” by S. G. Koolagudi et al., Int J Speech Technol 15:99-117, 2012 and “Deep Learning Techniques for Speech Emotion Recognition, from Databases to Models” by B. J. Abbaschian et al., Sensors 2021, 21, 1249).

### SUMMARY AND DESCRIPTION

The scope of the present disclosure is defined solely by the appended claims and is not affected to any degree by the statements within this description.

The present embodiments may obviate one or more of the drawbacks or limitations in the related art. For example, a method that enables a reduction in susceptibility to errors in medical interventions and examinations caused by stressful situations is provided. As another example, a system suitable for the performance of the method is provided.

In a method of the present embodiments for monitoring a medical intervention taking place in an operating room or an examination room, or a medical examination on a patient by medical personnel (e.g., with at least one medical device and/or at least one medical object), the following acts are performed: acquiring sensor data from at least one acoustic sensor (e.g., in the form of voice recordings) of at least one individual among the medical personnel during the intervention or the examination; evaluating the sensor data from the acoustic sensor as regards the recognition of stress and/or negative emotions of the at least one individual; when stress and/or negative emotions are recognized, determining at least one cause of the stress and/or of the negative emotions related to the intervention or the examination (e.g., using the sensor data and/or further optic, acoustic, haptic, digital, and/or other data acquired during the intervention or the examination); and triggering measures to eliminate the determined cause and/or to reduce the stress and/or the negative emotions of the at least one individual.

Thanks to the method, not only are the stress or the negative emotions of particular individuals among the medical personnel recognized by voice recordings, but a cause of the stress is also determined. For example, causes are included that are directly or indirectly related to the intervention or the examination. The cause is then selectively eliminated by initiating dedicated countermeasures. As a result, the stress or the negative emotion(s) may at least be reduced, and in the best case, even eliminated. Thanks to the method overall, medical interventions and examinations may therefore be made less stressful for the personnel. However, this also provides that the intervention or the examination becomes safer for the patient, since errors on the part of the personnel are significantly reduced. Both for the personnel and for the patient, the risk of health-related issues is therefore minimized. In addition, a medical intervention may also be accelerated thanks to the reduction in stress, which permits a higher patient throughput and better patient care.

One example of a negative emotion is anxiety or anger, which then contribute to stress. Tiredness or pain may also contribute to stress.

The medical intervention may, for example, involve a minimally invasive OP (e.g., navigation of a medical object (catheter, stent, guide wire, instrument, etc.) through a hollow organ of a patient with or without support from a robot system). The medical intervention may also involve 2D or 3D image acquisition by a medical device (e.g., an imaging device such as an X-ray device, CT, MR, ultrasound, etc.). This includes all medical examinations or interventions (e.g., also those with sequences consisting of multiple steps). Also included are interventions with remotely connected individuals among the medical personnel.

In accordance with one embodiment of the present embodiments, sensor data is acquired in the form of spoken language, and an evaluation of the spoken language is performed using at least one pretrained algorithm from machine learning and/or a database. Thanks to such algorithms, including, for example, deep learning algorithms or convolutional neural networks, stress or negative emotions may be recognized easily and especially quickly (e.g., live in the OP) from voice recordings. This is known, for example, from “Emotion recognition from speech: a review” by S. G. Koolagudi et al., Int J Speech Technol 15:99-117, 2012 and “Deep Learning Techniques for Speech Emotion Recognition, from Databases to Models” by B. J. Abbaschian et al., Sensors 2021, 21, 1249.

In accordance with a further embodiment, the cause of the stress and/or of the emotions is determined using the acoustic sensor data and/or sensor data from further sensors, system data from the medical device or other devices, medical imaging acquisitions of the patient, inputs by the individual, camera recordings of the intervention or of the examination, eye tracking data for the individual, vital parameters of the individual and/or of the patient, functional data of objects or devices and/or other information about the intervention or the examination. Thanks to at least one (e.g., multiple) of these information sources, a comprehensive analysis may be performed, and at least one cause of the stress or of the negative emotion(s) may be found. This above all includes causes that are directly or indirectly related to the intervention or the examination. The data used for the analysis may, for example, be transmitted from the corresponding sensors, devices, or information stores to a determination unit (e.g., by a wireless or wired data transmission path). The determination unit evaluates the corresponding data. At least one pretrained algorithm from machine learning may likewise, for example, be used for this. Besides the causes related to the intervention, general causes, such as, for example, tiredness of the individual, may additionally be taken into consideration.

In accordance with a further embodiment of the present embodiments, at least one of the following causes of the stress or of the negative emotions may be selected: problems with the medical device (e.g., imaging device; operational problems and/or errors that occur (device errors or user errors) and/or collisions of components or with other devices, problems with the medical object (operational problems) and/or malfunctions), problems with the intervention or the examination (e.g., errors and/or medical emergencies and/or unscheduled events that occur), and/or a conflict with another individual among the medical personnel.

In accordance with a further embodiment of the present embodiments, measures dependent on the cause of the stress or of the negative emotions are triggered. The corresponding measures are therefore specifically selected and implemented for the causes. In this way, the cause may be eliminated particularly effectively, and the stress or the negative emotion of the individual may be reduced.

In accordance with a further embodiment of the present embodiments, the measures to remediate the at least one determined cause also include at least one instance of operational support for the medical device or medical object (e.g., in the form of optical displays, automatic menu navigation, online help, checklists, or a simplified UI) and/or automatic assistance and/or automatic troubleshooting and/or collision management and/or automatic assistance with steps of the intervention or of the examination and/or a query to the individual.

The present embodiments also include a system for monitoring a medical intervention taking place in an operating room or examination room or a medical examination on a patient by medical personnel. The system includes at least one acoustic sensor for the acquisition of sensor data (e.g., of voice recordings) of at least one individual among the medical personnel, an evaluation unit for the evaluation of the sensor data as regards recognition of stress and/or negative emotions of the at least one individual, and a determination unit for the determination of at least one cause of the stress and/or of the negative emotions. The system also includes a control unit to trigger measures to remediate the determined cause and/or to reduce the stress and/or the negative emotions of the at least one individual. Thanks to the system, stress on the part of medical personnel may likewise be efficiently reduced, and thus, medical interventions or examinations may be performed more safely, faster, and more agreeably for everyone.

For example, the system has at least one operating unit for the operation of the system by the at least one individual among the medical personnel. This may, for example, involve a PC with an input unit and a display unit, or else a smart device or a joystick.

In one embodiment, for a particularly fast and comprehensive performance of the method, the evaluation unit and/or the determination unit has at least one trained algorithm for machine learning. Thanks to such algorithms, large volumes of data may be analyzed and processed especially quickly and easily, and selective results may be obtained.

The present embodiments also involve a complete medical system with a monitoring system. The complete medical system may include a medical device in the form of an imaging device (e.g., an X-ray device). This is used, for example, for radioscopy during the intervention or the examination. The complete medical system may include a robot system for the robot-based navigation of a medical object through a hollow organ of a patient.

The complete system may also include a plurality of other devices, objects, sensors, or measuring instruments. For data transmission and/or triggering, the monitoring system may be in data transmission connection with the other devices, objects, sensors, or measuring instruments (e.g., wirelessly (WLAN, Bluetooth, etc.) or wired).

In accordance with one embodiment of the present embodiments, at least one of the individuals among the medical personnel is participating in the intervention or the examination using a remote connection.

### BRIEF DESCRIPTION OF THE DRAWINGS

FIG. 1 shows a view of a complete system with a monitoring system in an operating room; and

FIG. 2 shows a method for monitoring a medical intervention taking place in an operating room or examination room.

### DETAILED DESCRIPTION

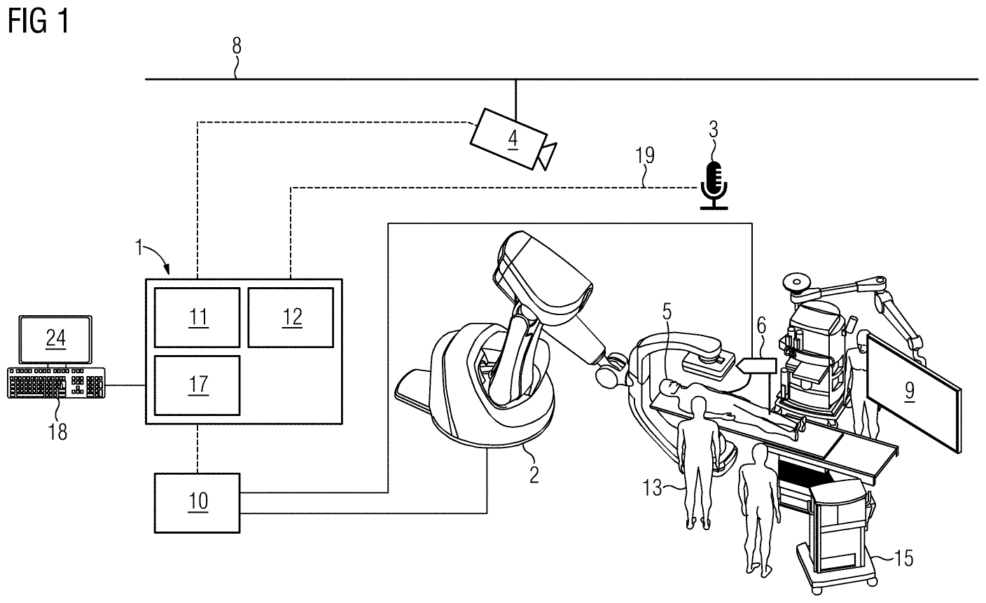

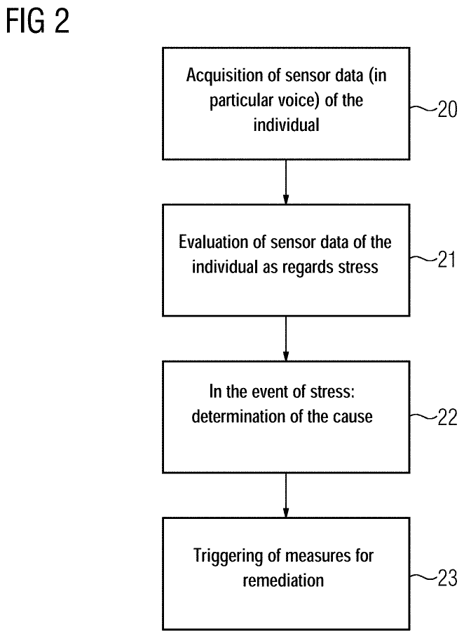

FIG. 1 shows a complete system with a monitoring system 1 for monitoring a medical intervention taking place in an operating room or an examination room 8, or a medical examination on a patient 5 by medical personnel. The acts of the associated method are shown in FIG. 2.

The medical intervention or the medical examination may involve any conceivable medical procedure. By way of example, navigation of a medical object (e.g., catheter, stent, guide wire, etc.) through a hollow organ of a patient using radiography is treated below. The complete system used for this has a monitoring system 1 with an evaluation unit 11, a determination unit 12, and a control unit 17. The complete system also includes an X-ray device 2 for acquisition of X-ray images (e.g., radioscopy images) and a robot system 6 for robot-based navigation of a medical object through a hollow organ of the patient 5. Components of the complete system may in part be arranged in a distributed manner across the operating room 8.

In a first act 20, voice recordings of at least one individual 13 among the medical personnel are acquired by an acoustic sensor during the corresponding intervention or the examination. The acoustic sensor may, for example, be formed by one microphone 3 or a plurality of microphones. The microphone 3 may be arranged in the operating room 8 in which the intervention is taking place or the individual 13 carries the microphone 3. The individual 13 may, for example, be a physician, a nurse, or a service technician. The individual 13 may be on site, and additionally, other medical personnel may be on site or connected remotely. The individual 13 may also be connected remotely, and other personnel may be on site.

In a second act 21, the sensor data (e.g., voice recordings) from the microphones 3 is evaluated as regards recognition of stress and/or negative emotions of the at least one individual. This is done, for example, by the evaluation unit 11. The voice recordings may first, for example, be transmitted from the microphone 3 to the evaluation unit 11 by a wireless or wired data transmission connection 19 (e.g., WLAN, Bluetooth, etc.).

Provision may be made for sensor data to be transmitted at specific points in time, at regular intervals or continuously, and to be evaluated live, in order to be able to respond quickly to any changes. The evaluation unit 11 may, for example, perform the evaluation using at least one pretrained algorithm from machine learning. Thanks to such algorithms (e.g., also including deep learning algorithms or convolutional neural networks (CNN, GAN, etc.)), stress and/or negative emotions may be recognized easily and especially quickly (e.g., thus, live in the OP) from the voice recordings. This is known, for example, from “Emotion recognition from speech: a review” by S. G. Koolagudi et al., Int J Speech Technol 15:99-117, 2012 and “Deep Learning Techniques for Speech Emotion Recognition, from Databases to Models” by B. J. Abbaschian et al., Sensors 2021, 21, 1249.

In addition, voice commands (e.g., for voice control of a device) may also be learned from the voice recordings.

One example of a negative emotion is anxiety or anger, for example, which may contribute to stress. Tiredness or pain may also contribute to stress.

In a third act 22, at least one cause of the stress and/or of the negative emotions is identified when stress and/or negative emotions are recognized (e.g., a cause such as that which is directly or indirectly related to the intervention or the examination). This is, for example, performed by a determination unit 12, where at least one pretrained algorithm from machine learning (e.g., machine learning, deep learning, CNN, GAN, etc.) may also be used for this.

In order to determine the cause(s), use may be made of the sensor data from the acoustic sensor (e.g., voice recordings from the microphones 3), and the spoken language contained therein may be analyzed (e.g., natural language processing (NLP); also with the aid of particular keywords). In addition, other optical, acoustic, haptic, digital, and/or other data acquired during the intervention or the examination that allows a conclusion to be drawn as regards the cause may be used to determine the cause.

A plurality of sources may be used for the data: other sensors or data acquisition units (e.g., microphones, cameras 4, haptic sensors, acceleration sensors, capacitive sensors, etc.); system data from the X-ray device, the robot system, or other devices (e.g., data about the operational sequence of the intervention, programs and parameters used, or error messages); medical imaging acquisitions of the patient (e.g., acquisitions acquired by the X-ray device) and evaluations of the acquisitions; inputs by the individual 13 or another individual among the medical personnel; eye tracking data for the individual 13; vital parameters of the patient 5 (e.g., EKG data, blood pressure or oxygen saturation, increased flow in external ventricle drainage, etc.); vital parameters of the individual 13; error messages of the system or individual devices; functional data and error messages of objects or devices (e.g., if an object does not behave as predicted, such as a stent does not open as predicted) or a collision has occurred, with/without damage to the device; temporal, spatial, or functional deviations from normal operational processes, sequences, and normal behavior of objects or individuals; time information (e.g., about the duration of steps of the intervention); and/or other information about the intervention or the examination. A request may also be issued to the individual to make an input (e.g., a question may be asked and the answer/input evaluated). The sensor data or other data may be transmitted by wireless or wired data transmission connections (e.g., WLAN, Bluetooth, etc.; dashed lines).

A plurality of possibilities may come into consideration as causes of stress. These possibilities include, for example: problems with the medical device (e.g., the X-ray device or robot system or contrast agent injector or other devices used for the intervention or the examination); problems with operation by the individual (e.g., an operating error, the individual requires assistance, cannot find functions, etc.); error of the device (e.g., error messages of the device) that occur; collisions of components of the device; problems with the medical object navigating in the body of the patient, inserted therein or treating the body of the patient (e.g., catheter, guide wire, device, instrument, etc.); operational problems on the part of the individual and/or malfunctions of the object; problems with the intervention or the examination (e.g., errors in the sequence and/or a medical emergency situation with respect to the patient and/or unscheduled events that occur during the intervention and/or excessive demands on the individual as regards actions required or decisions to be taken or an excessive duration of the intervention); a conflict or dispute with another individual among the medical personnel (e.g., because of different opinions, lack of agreement, or because some of the medical personnel are connected remotely); or any combination thereof.

If at least one cause is determined and found by the determination unit, then in a fourth act 23, at least one measure is triggered for the selective remediation of the determined cause. The control unit 17 may, for example, be provided for this. In addition, at least one general measure to reduce the stress and/or the negative emotions of the at least one individual may be triggered.

It is advantageous to match the measure directly to the cause. If, for example, the cause is due to an operational problem with respect to the X-ray device or robot system (e.g., operation places excessive demands on the individual, or the individual requires assistance, or cannot find functions), then, for example, simplifications in the user interface may be implemented automatically (e.g., the selection facilities for functional elements (buttons) are reduced to a necessary minimum, corresponding important functional elements are marked optically (in color, brighter, etc.), particular functional elements pertinent to the situation or “stress buttons” (cf. cardiopulmonary resuscitation, CPR button) are suggested). If, for example, an error message or a collision of components of the X-ray device (e.g., C-arm with patient table) or a blockage after a collision is recognized as a cause, then, for example, support may be offered automatically, for example, in unlocking the collision (e.g., online help may be suggested, step-by-step instructions may be overlaid, etc.).

If, for example, a problem with the intervention or the examination (e.g., errors in the sequence and/or a medical emergency situation with respect to the patient and/or unscheduled events that occur during the intervention and/or excessive demands on the individual as regards actions required or decisions to be taken) is recognized as a cause, then, for example, a detected error or an intended sequence of workflow steps of the intervention may be displayed automatically, detailed emergency checklists may be suggested and displayed, or support by another individual may be requested. Online helps, videos, online manuals, etc. matched to the respective situation may also be offered.

A selective request may also be made to the individual, and an input may be received and evaluated. For example, the measures to remediate the at least one determined cause may also include at least one instance of operational support in the case of the medical device (e.g., X-ray device 2 or robot system 6) or medical object (e.g., in the form of optical displays, automatic menu navigation, online help, checklists, or a simplified UI), and/or automatic assistance, and/or automatic troubleshooting, and/or collision management, and/or automatic assistance with steps of the intervention or of the examination, and/or a query to the individual.

The method may be performed throughout the entire intervention or else just during a selected time period. The method may be performed for a single individual or for multiple individuals involved in the intervention.

The monitoring system 1 also has an operating unit for the operation of the system by the at least one individual 13 among the medical personnel. This may, for example, be a PC with an input unit 18 and a monitor 24, or else a smart device or a joystick. The operating unit may be arranged in the operating room 8 or outside the operating room 8 or remotely. Further operating units (e.g., also by voice input) or display units 9 may be present in the operating room 8. The X-ray device 2 may, for example, be triggered by a system controller 10, and likewise, the robot system 6 may be triggered by a robot controller; alternatively, both are triggered by the same controller. The control unit 17 of the monitoring system may have a communication connection and a data exchange connection with the system controller 10. Provision may also be made for the complete system to be triggered by a common system control unit. The complete system may also include a plurality of other devices 15, objects, sensors, or measuring instruments.

The present embodiments may be summarized briefly as follows: for an especially risk-free, effective, and fast performance of medical interventions or examinations, a method is provided for monitoring a medical intervention taking place in an operating room or an examination room, or a medical examination on a patient by medical personnel (e.g., with at least one medical device and/or at least one medical object). The method includes acquiring sensor data from at least one acoustic sensor (e.g., in the form of voice recordings) of at least one individual among the medical personnel during the intervention or the examination, evaluating the sensor data from the acoustic sensor as regards recognition of stress and/or negative emotions of the at least one individual, and when stress and/or negative emotions are recognized, determining at least one cause of the stress and/or of the negative emotions (e.g., using the sensor data and/or further optical, acoustic, haptic, digital, and/or other data acquired during the intervention or the examination). The method includes triggering measures to remediate the determined cause and/or to reduce the stress and/or the negative emotions of the at least one individual.

While the present disclosure has been described in detail with reference to certain embodiments, the present disclosure is not limited to those embodiments. In view of the present disclosure, many modifications and variations would present themselves, to those skilled in the art without departing from the scope of the various embodiments of the present disclosure, as described herein. The scope of the present disclosure is, therefore, indicated by the following claims rather than by the foregoing description. All changes, modifications, and variations coming within the meaning and range of equivalency of the claims are to be considered within the scope.

It is to be understood that the elements and features recited in the appended claims may be combined in different ways to produce new claims that likewise fall within the scope of the present disclosure. Thus, whereas the dependent claims appended below depend from only a single independent or dependent claim, it is to be understood that these dependent claims may, alternatively, be made to depend in the alternative from any preceding or following claim, whether independent or dependent, and that such new combinations are to be understood as forming a part of the present specification.

1. A method for monitoring a medical intervention taking place in an operating room or an examination room, or a medical examination on a patient by medical personnel, the method comprising:
acquiring sensor data from at least one acoustic sensor, of at least one individual among the medical personnel during the medical intervention or the medical examination;
evaluating the sensor data from the at least one acoustic sensor as regards recognition of stress, negative emotions, or the stress and the negative emotions of the at least one individual;
when the stress, the negative emotions, or the stress and the negative emotions are recognized, determining at least one cause of the stress, the negative emotions, or the stress and the negative emotions using the sensor data, further optical data, further acoustic data, further haptic data, further digital data, other data acquired during the intervention or the examination, or any combination thereof; and
triggering measures, such that the determined at least one cause is remediated, the stress is reduced, the negative emotions of the at least one individual are reduced, or any combination thereof.

2. The method of claim 1, wherein the monitoring of the medical intervention takes place with at least one medical device, at least one medical object, or a combination thereof.

3. The method of claim 1, wherein the acquired sensor data is in the form of voice recordings.

4. The method of claim 1, wherein the sensor data is acquired in the form of spoken language, and
wherein the evaluating of the sensor data in the form of the spoken language is performed using at least one algorithm from machine learning, a database, or a combination thereof.

5. The method of claim 4, wherein the at least one cause of the stress, the emotions, or the stress and the emotions is determined using sensor data from further sensors, system data from medical devices or other devices, medical imaging acquisitions of the patient, inputs by the individual, camera recordings of the intervention or of the medical examination, eye tracking data for the individual, vital parameters of the individual, the patient, or the individual and the patient, functional data of objects or devices, other information about the intervention or the medical examination, or any combination thereof.

6. The method of claim 2, wherein the at least one determined cause of the stress, the negative emotions, or the stress and the negative emotions comprises: problems with the at least one medical device;
problems with the medical object;
problems with the medical intervention or the medical examination;
a conflict with another individual among the medical personnel; or
any combination thereof.

7. The method of claim 6, wherein the at least one determined cause of the stress, the negative emotions, or the stress and the negative emotions comprises problems with the at least one medical device, the problems with the at least one medical device including operational problems, errors that occur, collisions, or any combination thereof.

8. The method of claim 6, wherein the at least one determined cause of the stress, the negative emotions, or the stress and the negative emotions comprises problems with the medical intervention or the medical examination, the problems with the medical intervention or the medical examination including errors, medical emergencies, unscheduled events that occur, or any combination thereof.

9. The method of claim 1, wherein triggering measures comprises triggering measures dependent on the at least one determined cause of the stress, the negative emotions, or the stress and the negative emotions.

10. The method of claim 2, wherein triggering measures comprises triggering measures, such that the determined at least one cause is remediated, and
wherein the measures include at least one instance of:
operational support with the at least one medical device, the at least one medical object, or the at least one medical device and the at least one medical object;
automatic assistance;
automatic troubleshooting;
collision management;
automatic assistance with steps of the medical intervention or of the medical examination;
a query to the at least one individual; or
any combination thereof.

11. The method of claim 10, wherein the operational support with the at least one medical device, the at least one medical object, or the at least one medical device and the at least one medical object includes optical displays, automatic menu navigation, online help, checklists, or a simplified user interface (UI).

12. The method of claim 1, wherein one or more individuals of the at least one individual among the medical personnel is taking part in the medical intervention or the medical examination using a remote connection.

13. A system for monitoring a medical intervention taking place in an operating room or an examination room, or a medical examination on a patient by medical personnel, the system comprising:
at least one acoustic sensor configured to acquire sensor data from at least one individual among the medical personnel;
an evaluation unit configured to evaluate the sensor data as regards recognition of stress, negative emotions, or the stress and the negative emotions of the at least one individual;
a determination unit configured to determine at least one cause of the stress, the negative emotions, or the stress and the negative emotions; and
a control unit configured to trigger measures, such that the determined at least one cause is remediated, the stress is reduced, the negative emotions of the at least one individual is reduced, or any combination thereof.

14. The system of claim 13, further comprising at least one operating unit configured for operation of the system by the at least one individual among the medical personnel.

15. The system of claim 13, wherein the evaluation unit, the determination unit, or the evaluation unit and the determination unit have at least one trained algorithm for machine learning.

16. A complete medical system comprising:
a monitoring system for monitoring a medical intervention taking place in an operating room or an examination room, or a medical examination on a patient by medical personnel, the monitoring system comprising:
at least one acoustic sensor configured to acquire sensor data from at least one individual among the medical personnel;
an evaluation unit configured to evaluate the sensor data as regards recognition of stress, negative emotions, or the stress and the negative emotions of the at least one individual;
a determination unit configured to determine at least one cause of the stress, the negative emotions, or the stress and the negative emotions; and
a control unit configured to trigger measures, such that the determined at least one cause is remediated, the stress is reduced, the negative emotions of the at least one individual is reduced, or any combination thereof.

17. The complete medical system of claim 16, further comprising:
a medical device in the form of an imaging device.

18. The complete medical system of claim 17, wherein the imaging device comprises an X-ray device.

19. The complete medical system of claim 16, further comprising a robot system for robot-based navigation of a medical object through a hollow organ of the patient.

20. The complete medical system of claim 16, wherein one or more individuals of the at least one individual among the medical personnel is taking part in the medical intervention or the medical examination using a remote connection.
